(19) United States
(12) Patent Application Publication
(10) Pub. No.: US 2021/0087128 A1
(43) Pub. Date: Mar. 25, 2021

(54) PROCESS WITH INTEGRATED RECYCLE FOR MAKING ETHYLENE GLYCOL AND/OR PROPYLENE GLYCOL FROM ALDOSE- AND/OR KETOSE- YIELDING CARBOHYDRATES

(71) Applicant: Iowa Corn Promotion Board, Johnston, IA (US)

(72) Inventors: David James Schreck, Lake City, MN (US); Ray Chrisman, Midland, MI (US); Donald Bunning, South Charleston, WV (US); Lou Kapicak, Cross Lanes, WV (US); Brooke Albin, Charleston, WV (US); Mark Nunley, Charleston, WV (US); Michael Bradford, Charleston, WV (US)

(21) Appl. No.: 17/031,510

(22) Filed: Sep. 24, 2020

Related U.S. Application Data
(60) Provisional application No. 62/904,854, filed on Sep. 24, 2019.

(51) Int. Cl. C07C 29/17

(52) U.S. Cl. CPC C07C 29/172

(57) ABSTRACT

Processes are disclosed for the catalytic conversion of carbohydrate feed to one or both of ethylene glycol and propylene glycol. In the disclosed processes, a portion of the aqueous medium in the reaction zone of the catalytic process is withdrawn and recycled and the recycle is integrated to enhance the overall process.

### CROSS-REFERENCE TO RELATED APPLICATION(S)

This application claims the benefit under 35 U.S.C. § 119(e) to U.S. Provisional Application 62/904,854 filed Sep. 24, 2019 and entitled “PROCESS WITH INTEGRATED RECYCLE FOR MAKING ETHYLENE GLYCOL AND/OR PROPYLENE GLYCOL FROM ALDOSE- AND/OR KETOSE-YIELDING CARBOHYDRATES,” which is hereby incorporated herein by reference in its entirety for all purposes.

### TECHNICAL FIELD

This invention pertains to processes for the production of ethylene glycol and/or propylene glycol from aldose- and/or ketose-yielding carbohydrates, particularly processes that have an integrated recycle.

### BACKGROUND

Ethylene glycol and propylene glycol are valuable commodity chemicals and each has a broad range of uses. These chemicals are currently made from starting materials based upon fossil hydrocarbons (petrochemical routes).

Proposals have been made to manufacture ethylene glycol and propylene glycol from renewable resources such as carbohydrates, e.g., sugars. One such route has been practiced commercially and involves the fermentation of sugars to ethanol, catalytically dehydrogenating the ethanol to ethylene and the ethylene is then catalytically converted to ethylene oxide which can then be reacted with water to produce ethylene glycol. This route is not economically attractive as three conversion steps are required, and it suffers from conversion efficiency losses. For instance, the theoretical yield of ethanol is 0.51 grams per gram of sugar with, on a theoretical basis, one mole of carbon dioxide being generated per mole of ethanol.

Alternative processes to make ethylene glycol and propylene glycol from renewable resources are thus sought. These alternative processes include catalytic routes such as hydrogenolysis of sugar and a two-catalyst process using a retro-aldol catalyst to generate intermediates from sugar that can be hydrogenated over a hydrogenation catalyst to produce ethylene glycol and propylene glycol. The former process is referred to herein as the hydrogenolysis process or route, and the latter process is referred to as the hydrogenation, or retro-aldol, process or route. For the sake of ease of reference, the latter is herein referred to as the retro-aldol process or route. The term “catalytic process” or “catalytic route” is intended to encompass both hydrogenolysis and the retro-aldol route.

In the catalytic routes, carbohydrate (which may be one carbohydrate or a mixture of carbohydrates) that yields aldose or ketose, is passed to a reaction zone containing catalyst in an aqueous medium. At elevated temperature and the presence of hydrogen, the carbohydrate is converted to ethylene glycol and/or propylene glycol. The hydrogenolysis process uses a hydrogenolysis catalyst, and typically temperatures below about 225° C. In many instances, high conversions of the carbohydrate can occur at temperatures below about 220° C. The hydrogenolysis route often uses a low concentration of carbohydrate fed to the reaction zone to attenuate the production of by-products. The retro-aldol route is fundamentally different in that the carbohydrate is converted over a retro-aldol catalyst to intermediates, and then the intermediates are then catalytically converted over a hydrogenation catalyst to ethylene glycol and/or propylene glycol. The sought initially-occurring retro-aldol reaction is endothermic and requires a high temperature, e.g., often over 230° C., to provide a sufficient reaction rate to preferentially favor the conversion of carbohydrate to intermediates over the hydrogenation of carbohydrate to polyol such as sorbitol.

Over time, laboratory-scale, catalytic processes to convert carbohydrates to ethylene glycol and propylene glycol, and especially the retro-aldol route, have evidenced improvements in selectivity and conversion efficiency. These improvements have now given cause to consider the manner in which the catalytic routes should be implemented to provide a commercial-scale facility that could be competitive with the petrochemical routes to make these chemicals.

Both catalytic routes, by their very nature, present a myriad of complexities that affect the economics of a commercial facility, both in capital and operating expenses. Accordingly, a desire exists to develop catalytic processes that can be cost-effective on a commercial-scale.

### BRIEF SUMMARY

By this invention, catalytic processes are provided that can enhance the economics of producing ethylene glycol and/or propylene glycol from carbohydrates. In the processes of this invention, a portion of the media in the reaction zone of the catalytic process is withdrawn, subjected to at least one unit operation and at least a portion of the withdrawn media is recycled, and the recycle is integrated with the operation of the process. In some instances, the recycle is provided or maintained at elevated temperatures to provide conservation of heat energy for the process.

### First Broad Aspect of this Invention

In a first aspect of the processes of this invention, the catalytic process is effected in a reactor that contains catalyst that is dissolved or suspended in an aqueous reaction medium. At least a portion of the aqueous medium is withdrawn from the reactor for the recovery of ethylene glycol and propylene glycol, and the withdrawn aqueous medium contains at least one catalyst. A catalyst that is withdrawn can be in the form of one or more of a dissolved species or a suspended solid. In accordance with this first broad aspect of the invention, the withdrawn aqueous medium is subjected to one or more separation unit operations, preferably including a vapor/liquid separation (as defined herein), whereby only a portion of the ethylene glycol and propylene glycol passes to a separated fraction, which may, for instance be an extracted fraction in the case of an extraction unit operation, a sorbed or non-sorbed fraction in the case of sorption, a permeate in the case of a membrane separation, and a vapor phase in the case of a vapor/liquid separation and at least about 10, say, 25 to 75, and in some instances from about 35 to 65, mole percent of the total ethylene glycol and propylene glycol of that amount contained in the withdrawn aqueous phase, remains in a retained, liquid phase as does the catalyst. The remaining ethylene glycol and propylene glycol may be in the same or different mole ratio than their mole ratio in the withdrawn aqueous phase. At least an aliquot or aliquant portion of this liquid phase is recycled to the reactor. Without wishing to be limited by theory, it is believed that by retaining a sufficient amount of one or both of ethylene glycol and propylene glycol in the liquid phase from the separation unit operations, undue adverse effects on the withdrawn catalyst are attenuated. The adverse effects can be physical or chemical in nature. Thus, the catalyst can effectively be recycled to the reaction zone without undue loss of activity. In some instances the mass ratio of catalyst in the liquid phase from the separation unit operations to lower glycol is from about 0.01:1 to 10:1, preferably 0.05:1 to 2:1. Often the liquid phase will contain some water, say, up to about 10, and sometimes from 0.1 to 5, volume percent. Preferably the separation unit operations serve to remove at least a portion of the by-product organic acids generated by the catalytic process. The remaining liquid phase from the separation unit operations will typically also contain higher boiling coproducts from the catalytic conversion such as sorbitol and glycerin. In some instances, hydrogenolysis or hydrogenation conditions in the reactor can convert these higher boiling compounds to lower glycols. Hence recycling of these higher boiling compounds can be implemented. In some instances, separation of these higher boiling compounds from the liquid medium, e.g., by simulated moving bed chromatography, can reduce the amount of the liquid medium that is purged to maintain steady-state operation of the process.

This broad aspect pertains to catalytic processes for producing a lower glycol comprising at least one of ethylene glycol and propylene glycol from a carbohydrate-containing feed that comprises at least one of aldose- and ketose-yielding carbohydrate, said processes comprising continuously or intermittently supplying the feed to a reaction zone containing an aqueous medium having therein one or more catalysts for converting said carbohydrate to said glycol, wherein at least one of the catalysts is dissolved or suspended in the aqueous medium, said aqueous medium being at catalytic conversion conditions including the presence of dissolved hydrogen, to produce a reaction product containing said lower glycol, wherein
(i) continuously or intermittently at least a portion of the aqueous medium containing said dissolved or suspended catalyst is withdrawn from the reaction zone;
(ii) at least a portion of the withdrawn aqueous medium is subjected to one or more unit operations, preferably at least one vapor/liquid separation unit operation, to remove a portion of the lower glycol in a separated fraction and provide a retained liquid phase containing at least about 10 mass percent of the lower glycol contained in the aqueous medium as withdrawn from the reaction zone and said dissolved or suspended catalyst; and
(iii) at least a portion of the retained liquid phase containing the dissolved or suspended catalyst from the one or more unit operations is passed to the reaction zone.

In one embodiment of this first broad aspect, the reaction product contains organic acid, and at least about 10, preferably at least about 25, and sometimes from about 30 to 70 or 90, mass percent of the organic acid is passed to the separated fraction, e.g., to the vapor phase where the unit operation is a vapor/liquid separation.

In another embodiment of this first broad aspect, the one or more unit operations is a vapor/liquid separator and water is added to at least the portion of the retained liquid phase passed to the reaction zone to provide a recycle liquid comprising at least about 10 or 25, and sometimes at least about 35, mass percent water. Where the lower glycols are separated from the liquid withdrawn from the reaction zone by vapor/liquid separation, often the mass ratio of total lower glycol to water in the remaining liquid phase is at least about 20:1, and sometimes at least about 50:1 or 100:1. To the liquid phase being recycle can be added one or more other components including, but not limited to, carbohydrate; catalyst for the catalytic conversion; pH modifiers; hydrogen; and adjuvants such as additives and reactants to enhance catalyst stability and, in the case of homogeneous retro-aldol catalyst enhance solubility.

In another embodiment of this first broad aspect, the catalytic process embodies the retro-aldol route and the retained liquid phase contains the retro-aldol catalyst. Preferably at least a portion of the retro-aldol catalyst is passed with the recycle liquid phase to the reaction zone. The retro-aldol catalyst being recycled can be subjected to one or more unit operations to restore or enhance the activity of the catalyst.

The recycle liquid phase can be admixed with at least a portion of the carbohydrate supplied to the reaction zone or separately introduced into the reaction zone. The liquid phase from the one or more separation unit operations will typically have a hydrogen partial pressure lower than that of the aqueous medium in the reaction zone, often less than about 1000, preferably less than 500, kilopascal, and where the unit operation comprises a vapor/liquid separation, often very little hydrogen would be retained, until the recycle liquid.

In one embodiment of this first broad aspect, the recycled liquid phase has no hydrogen added prior to being introduced into the reaction zone. In a preferred embodiment of this first broad aspect, at least one of carbohydrate being supplied and hydrogen being supplied to the reaction zone are combined with the portion of the liquid phase being passed to the reaction zone. In some instances, the liquid phase as it is being passed to the reaction zone contains hydrogen and is under hydrogenolysis conditions, including the presence of hydrogenolysis catalyst, such that at least a portion of the carbohydrate is catalytically converted to ethylene glycol and propylene glycol prior to introduction into the reaction zone. In such embodiments, frequently, the concentration of the carbohydrate (on an anhydrous basis) in the recycle liquid phase is less than about 500, often less than about 350, preferably less than about 300, grams per liter of recycle liquid phase.

Where the retro-aldol route is being used, especially if the recycle liquid phase contains little, if any, hydrogenation catalyst, hydrogen can be introduced into the recycle liquid phase to supply hydrogen for the hydrogenation of intermediates formed by the retro-aldol reactions. In some instances, the liquid phase can be used as the motive liquid for an eductor, or injector, to supply hydrogen to the reaction zone.

If desired, make-up or fresh catalyst (hydrogenolysis catalyst for the hydrogenolysis route or at least one of retro-aldol catalyst and hydrogenation catalyst for the retro-aldol route) for the catalytic processes can be introduced directly or indirectly into the reaction zone. Where indirectly introduced, catalyst is often admixed with the recycle liquid phase prior to its introduction into the reaction zone and/or admixed with the feed prior to its introduction into the reaction zone.

In a further embodiment of this first broad aspect, at least a portion of the retained liquid phase from the one or more separation unit operations is continuously or intermittently removed as a liquid phase purge. Often, the purge rate is sufficient to maintain the pH of the aqueous medium within a sought range, say, within a pH range of +/−2, and preferably +/−1.5 pH units, of the targeted range. For the hydrogenolysis route, the targeted pH often is in the range of about 5 to 9 or 12, say, about 6 to 8 or 11, and for the retro-aldol route, in the range of about 3 to 8, frequently about 3 or 3.5 to 7, say, 3.5 or 4 to 6.5.

The process of this first broad aspect provides a retained liquid phase from the separation unit operations that contains lower glycol and heavier organics such as glycerin and sorbitol. In the retro-aldol process, especially where tungsten-containing compound is used as the homogeneous retro-aldol catalyst, precipitates from the retro-aldol catalyst onto the hydrogenation catalyst can occur and result in a loss of hydrogenation activity. The concentration of lower glycol in the retained liquid phase together with reduced water content, sometimes results in at least a portion of the precipitates being solubilized. Removal of deposits can also be accomplished by increasing the pH, e.g., to greater than about 4.5. At these pH's, the solubilized tungsten compound is believed to convert into a species that has greater solubility in water. At least a portion of the liquid phase that has been pH adjusted, can be returned to the reaction zone. The solubilized tungsten compound is believed to be catalytically active or forms a catalytically active species, thereby conserving tungsten. The pH adjustment is frequently to from about 4 or 4.5 to 10, say, from about 4 or 5 to 6 or 9.

These catalytic processes for producing a lower glycol of at least one of ethylene glycol and propylene glycol from a carbohydrate-containing feed comprising at least one of aldose- and ketose-yielding carbohydrate, said processes comprise continuously or intermittently supplying the feed to a reaction zone containing an aqueous medium having therein a homogeneous, tungsten-containing retro-aldol catalyst and heterogeneous hydrogenation catalyst for converting said carbohydrate to said glycol, said aqueous medium being at catalytic conversion conditions including the presence of dissolved hydrogen, to produce a reaction product containing said lower glycol, wherein
(i) tungsten compound precipitates on the hydrogenation catalyst during the process;
(ii) continuously or intermittently at least a portion of the aqueous medium containing said hydrogenation catalyst is withdrawn from the reaction zone;
(iii) at least a portion of the withdrawn aqueous medium containing hydrogenation catalyst is subjected to vapor/liquid separation to remove water and a portion of the lower glycol in the vapor phase and provide a liquid phase containing at least about 10 mass percent of the lower glycol contained in the aqueous medium as withdrawn from the reaction zone and less than 10 volume percent water, and said hydrogenation catalyst wherein at least of the portion of the tungsten compound precipitated on the hydrogenation catalyst is solubilized;
(iv) optionally, water is added to the liquid phase containing lower glycol, and the pH is maintained from about 4 to 10 to maintain solubilized tungsten compound; and
(v) at least a portion of the liquid phase containing the solubilized tungsten compound is passed to the reaction zone.

### Second Broad Aspect of the Invention

The second broad aspect of this invention pertains to facilitating long-term, continuous catalytic processes for making lower glycol from carbohydrate in which processes organic acid is formed as a byproduct and at least a portion of the organic acid formed is removed from a recycle stream. The removal of organic acid assists in maintaining a desired pH during the catalytic reaction. It should be understood that the process can comprise other unit operations directed to maintaining a desired pH in the aqueous medium in the reaction zone. For instance, base or buffer can be present or added to the reaction zone and/or base or buffer can be added to the recycle stream for pH control. Continuous or intermittent addition of base or buffer, however, could necessitate a high purge rate in the continuous process. A purge results in losses of lower glycols that are not recovered from the recycle stream prior to the purge. It may be desired to recover additional lower glycols from the purge by suitable unit operations as are known in the art. Moreover, especially with the retro-aldol route, loss of retro-aldol catalyst occurs with an economic penalty to the process either in disposal with the purge or in costs to recover catalyst from the purge.

This second broad aspect pertains to catalytic processes for producing a lower glycol comprising at least one of ethylene glycol and propylene glycol from carbohydrate that is at least one of aldose- and ketose-yielding carbohydrate comprising continuously or intermittently supplying the carbohydrate to a reaction zone containing an aqueous medium having therein catalyst for converting said carbohydrate to said glycol, said aqueous medium being at catalytic conversion conditions including the presence of dissolved hydrogen, to produce a reaction product containing said lower glycol and organic acid, wherein
continuously or intermittently at least a portion of the aqueous medium is withdrawn from the reaction zone;
at least a portion of the withdrawn aqueous medium is subjected to vapor/liquid separation sufficient to remove at least about 25, sometimes at least about 35, and preferably at least about 50, mass percent of the organic acid contained in the withdrawn aqueous medium and provide a liquid phase; and
at least a portion of the liquid phase from the vapor/liquid separation is passed to the reaction zone.

In one embodiment of this second aspect of the invention, the one unit operation comprises a vapor/liquid separation providing a vapor phase that removes a portion of the lower glycol to the vapor phase and at least about 35 mass percent of the organic acid is separated into the vapor phase.

In one embodiment of this second aspect of the invention, a portion of the liquid phase passing to the reaction zone is purged, and the vapor/liquid separation and purge rate are sufficient to maintain the pH of the aqueous medium withdrawn from the reaction zone before being subjected to the vapor/liquid separation, within a sought range, say, within a pH range of +/−2, and preferably +/−1.5 pH units, of the targeted range. For the hydrogenolysis route, the targeted pH often is in the range of about 5 to 9 or 12, say, about 6 to 8 or 11, and for the retro-aldol route, in the range of about 3 to 8, frequently about 3 or 3.5 to 7, say, 3.5 or 4 to 6.5.

In one embodiment of this second broad aspect of the invention, the organic acid comprises at least one of acetic acid or dimer thereof and glycolic acid.

### Third Broad Aspect of the Invention

The third broad aspect of this invention pertains to facilitating long-term, continuous catalytic processes for making lower glycol from carbohydrate. During the continuous process particulate solids, which solids are often less than one micron in major dimension, can be generated via a number of routes. For instance, particulate solids can form when heterogeneous catalysts physically degrade. Homogeneous catalysts can precipitate when reacted with a counter ion or otherwise form a species that precipitate. In some instances, small particulates may be added to the reaction zone as catalysts, precursors to catalysts (such as where tungstic acid is used as a precursor to a retro-aldol catalyst), or adjuvant.

In this third aspect of the invention, a purge is taken continuously or intermittently from a recycle stream, and the purge rate is sufficient to maintain the concentration of particulate solids in the withdrawn aqueous medium from the reaction zone substantially constant. By substantially constant, the concentration can vary within a range of from about +/−20, to preferably +/−10, percentage points.

This third broad aspect of this invention pertains to catalytic processes for producing a lower glycol comprising at least one of ethylene glycol and propylene glycol from carbohydrate feed comprising at least one of aldose- and ketose-yielding carbohydrate comprising continuously or intermittently supplying the carbohydrate feed to a reaction zone containing an aqueous medium having therein catalyst for converting said carbohydrate to said glycol, said aqueous medium being at catalytic conversion conditions including the presence of dissolved hydrogen, to produce a reaction product containing said lower glycol and wherein particulate solids are generated, wherein
continuously or intermittently at least a portion of the aqueous medium is withdrawn from the reaction zone;
at least a portion of the withdrawn aqueous medium is subjected to one or more separation unit operations, preferably comprising a vapor liquid separation, to remove at least a portion of the lower glycol and provide a remaining liquid phase;
a portion of the liquid phase is passed to the reaction zone, and
continuously or intermittently a portion of the liquid phase is purged to maintain the particulate solids concentration in the aqueous medium substantially constant.

In a further embodiment of this third aspect of the invention, the purge is subjected to one or more unit operations to recover catalytic metals from the purge. The catalytic metals are components of the hydrogenolysis catalyst, hydrogenation catalyst and retro-aldol catalyst. One such unit operation is ion exchange, and sometimes cation exchange or anion exchange. Another such unit operation is filtration to recover particulates including any precipitates of components from the catalysts or supports. Particulates can also be recovered via density separation, e.g., settling, vane separation, hydrocyclone separation or centrifugation. The purge may be subjected to a sorption unit operation to remove metals, e.g., using activated carbon. The purge may be subjected to chemical treatment to cause precipitation of metal containing ions, which can be cations or anions, generated by the catalysts. This treatment includes, but is not limited to, (i) introducing counter ions to precipitate or (ii) causing an oxidation or reduction of, the metal containing ions into a solid. For instance, tungsten-containing ions that are or are derived from retro aldol catalyst used in the retro aldol route can be acidified to form less soluble tungstic acid that results in precipitates for recovery by, for example, filtration. Magnetic separation is yet another method for recovery of hydrogenolysis catalyst or hydrogenation catalyst components such as nickel and other metals that are magnetic. In some instances, separations are enhanced by the addition of coagulants or flocculants such as polymeric agents although inorganic agents such as alum can be used but it is preferred that the aqueous medium returning to the reaction zone be substantially free of such coagulants or flocculants. Simulated moving bed chromatography can be useful for recovery of dissolved catalytic metals from the hydrogenolysis or hydrogenation catalyst and especially the homogeneous retro-aldol catalyst.

### Fourth Broad Aspect of the Invention

A fourth broad aspect of this invention pertains to subjecting at least a portion of the aqueous medium withdrawn from the reaction zone to one or more unit operations to enhance the process such as, and without limitation, to recover catalyst, to regenerate catalysts, and to remove by other than a purge, undesired components generated during the catalytic conversion or introduced with feedstocks, and then recycling the aqueous medium to the reaction zone.

This fourth broad aspect of the invention pertains to catalytic processes for producing a lower glycol, that is, at least one of ethylene glycol and propylene glycol, from carbohydrate comprising at least one of aldose- and ketose-yielding carbohydrate comprising continuously or intermittently supplying the carbohydrate feed to a reaction zone containing an aqueous medium having therein catalyst for converting said carbohydrate to said glycol, said aqueous medium being at catalytic conversion conditions including the presence of dissolved hydrogen, to produce a reaction product containing said glycol, wherein
(a) continuously or intermittently at least a portion of the aqueous medium is withdrawn from the reaction zone, and
(b) at least a portion of the withdrawn aqueous medium from the reaction zone is recycled to the reaction zone,
wherein in step (b) the aqueous medium recycling to the reaction zone is subjected to at least one unit operation to enhance the catalytic process in the reaction zone.

The portion of the withdrawn aqueous medium that is recycled to the reaction zone can be an aliquot or aliquant portion. Where an aliquant portion, that is the concentration of components in the portion of the aqueous medium being recycled is different from that of the withdrawn aqueous medium. Aliquant portions would occur when the aqueous medium withdrawn is subjected to vapor/liquid separation, a filtration or other unit operation that selectively reduces concentration of one or more components of the aqueous medium as withdrawn from the reaction zone.

In one embodiment of this fourth broad aspect of the invention, the withdrawn aqueous medium is not subjected to separation unit operations to remove lower glycol, but rather serves to enable continuous or intermittent unit operations to occur outside the reaction zone. In another embodiment the withdrawn aqueous medium is subjected to one or more unit operations, preferably comprising vapor/liquid separation, to remove lower glycol and provide a retained liquid phase and at least a portion of the retained liquid phase is recycled to the reaction zone.

In one embodiment of this fourth broad aspect of the invention, the aqueous medium withdrawn from the reaction zone contains catalytic metals and the unit operation to enhance the catalytic process in the reaction zone is a removal of at least a portion of the catalytic metals contained in the withdrawn aqueous medium. The catalytic metals may, for instance, be components of the hydrogenolysis or hydrogenation catalyst that have been adversely affected by dissolution or physical degrading. The catalytic metals for the retro-aldol process would include dissolved or precipitated compounds or complexes of the metal of the retro-aldol catalyst. Such compounds or complexes could include the retro-aldol catalyst and other compounds or complexes derived from the retro-aldol catalyst under the conditions in the reaction zone. Fresh catalyst can be added to the reaction zone to reflect loss of catalytic metals by the removal unit operation. By removing catalytic metals, the catalytic process in the reaction zone is enhanced via one or more routes such as removing less active or inert catalytic metals enabling replacement by active catalyst.

The unit operation to remove catalytic metals can be composed of one or more unit operations. Unit operations include, but are not limited to, membrane separation, sorption, filtration and density-based separations such as centrifugation, cyclonic separation, and gravity settling. Where the catalytic metals are dissolved, the removal can be by any suitable unit operation such as membrane separation, sorption, magnetic separation if metal particles exist at are attracted by magnetic forces, ion exchange and chemical reaction to precipitate such dissolved metals followed by particle removal. In some instances, separations are enhanced by the addition of coagulants or flocculants such as polymeric agents although inorganic agents such as alum can be used but it is preferred that the aqueous medium returning to the reaction zone be substantially free of such coagulants or flocculants. Simulated moving bed chromatography can be useful for recovery of dissolved catalytic metals from the hydrogenolysis or hydrogenation catalyst and especially the homogeneous retro-aldol catalyst.

In one mode of removal of catalytic metal, the catalyst for converting the carbohydrate to glycol comprises a retro-aldol catalyst and the retro-aldol catalyst is a soluble tungsten-containing catalyst that is, or is converted during the process to, a tungsten-containing anion that can be converted to tungstic acid at low pH. In the unit operation, the pH of the aqueous medium is sufficiently reduced that tungstic acid is precipitated and then removed by a solids separation unit operation. Often the pH of the aqueous medium is lowered to less than about 3, preferably less than about 2. The removed tungstic acid can, if desired, be regenerated by reacting the tungstic acid with base to form a soluble tungstate anion. The preferred base is alkali metal base, especially sodium hydroxide. In another mode of removal of tungsten-containing anion, the medium is contacted with a sorbent for the tungstate such as activated carbon.

In another embodiment of this fourth broad aspect of the invention, the recycling aqueous medium is treated to enhance or regenerate the catalyst. In one mode of this embodiment pertains to the retro-aldol route where a soluble tungsten-containing catalyst is used, and solid, less active or inactive tungsten-containing species form in the reaction zone. In this mode, the aqueous medium being recycled to the reaction zone is subjected to a unit operation to convert tungsten-containing species to active or more active tungsten-containing species. One such unit operation is an oxidation. Any suitable oxidant can be used such as oxygen, ozone, peroxides, e.g., hydrogen peroxide, hydroperoxides, peroxyacids, diacyl peroxides, dialkyl peroxides, such as peracetic acid, and soluble peracid and peroxyanion compounds such as peroxycarbonate, perchlorate and permanganate. Hydrogen peroxide is preferred.

In another embodiment of this fourth broad aspect of the invention, the unit operation to enhance the catalytic process in the reaction zone involves the hydrogenation of organic acids. Organic acids are sometimes contained in the feedstock used in the processes and additionally organic acids can be generated during the catalytic processes. The hydrogenolysis route and the retro-aldol route usually use catalysts and conditions that are not so severe that organic acid groups are hydrogenated. By this embodiment, at least a portion of the withdrawn aqueous medium is subjected to carboxylic acid hydrogenation conditions including the presence of a carboxylic acid hydrogenation catalyst and hydrogen at elevated temperature and pressure. At least a portion of the carboxyl groups are converted to hydroxyls. Preferably, the absolute amount of organic acid is reduced by at least about 25, and more preferably by at least about 50, mass percent. Examples of carboxylic acid reducing catalytic metals are copper, platinum and ruthenium. Preferably the carboxylic acid reducing catalyst is supported to facilitate separation from the aqueous medium. Supports for the carboxylic acid reducing catalyst include, but are not limited to, activated carbon; silica; silica alumina; alumina such as gamma, transition aluminas and alpha alumina; zirconia; titania; and ceria. Carboxylic acid hydrogenation conditions include temperatures of from about 150° C. to 300° C. and hydrogen partial pressures of from about 2000 to 50,000, often from about 4000 to 25,000, kilopascals.

### Fifth Broad Aspect of the Invention

In accordance with the fifth broad aspect of the invention, aqueous medium that contains heterogeneous hydrogenation or hydrogenolysis catalyst is withdrawn from the reaction zone, and at least a portion of this withdrawn aqueous medium is recycled with the heterogeneous catalyst to the reaction medium. Prior to being introduced into the reaction zone, hydrogen is introduced into the recycling aqueous medium. Preferably, the recycled aqueous medium is introduced into the reaction zone in a manner to facilitate mixing of the heterogeneous catalyst in the reaction zone.

As the solubility of hydrogen in aqueous media is low, achieving adequate mass transfer of hydrogen to the hydrogenation catalyst can be challenging. Introducing hydrogen into the recycle stream prior to contacting carbohydrate in the case of hydrogenolysis or intermediates in the case of the retro-aldol route, assures hydrogen is present proximate to the heterogeneous catalyst when the hydrogenolysis or hydrogenation is commenced. In some instances, the recycling aqueous medium can be subjected to sufficient hydrogen partial pressure to facilitate hydrogenolysis and hydrogenation by allowing the surface of the catalyst, especially the catalytic metals, to become laden with hydrogen.

Accordingly, this fifth broad aspect of the invention pertains to catalytic processes for producing a lower glycol which is at least one of ethylene glycol and propylene glycol from carbohydrate feed that comprises at least one of aldose- and ketose-yielding carbohydrate comprising continuously or intermittently supplying the carbohydrate feed to a reaction zone containing an aqueous medium having therein catalyst for converting said carbohydrate to said glycol, said catalyst comprising heterogeneous hydrogenation or hydrogenolysis catalyst, and said aqueous medium being at catalytic conversion conditions including the presence of dissolved hydrogen, to produce a reaction product containing said glycol, wherein
continuously or intermittently at least a portion of the aqueous medium is withdrawn from the reaction zone, said withdrawn aqueous medium containing heterogeneous catalyst, and
recycling at least a portion of the withdrawn aqueous medium from the reaction zone to the reaction zone, said recycled aqueous medium containing heterogeneous catalyst from the reaction zone, and prior to being introduced into the reaction zone, hydrogen is admixed with the recycling aqueous medium.

Often the hydrogen supplied provides a partial pressure of from about 2000 to 50,000, often from about 4000 to 25,000, kilopascals. The hydrogen-laden aqueous medium can be supplied to the reaction zone. In some instances, the hydrogen-laden aqueous medium is maintained for a duration and a temperature sufficient to reduce at least a portion of the oxidized species of the catalytic metal of said heterogeneous catalyst.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a schematic depiction of an apparatus that can be used to practice the processes of this invention.

FIG. 2 is a schematic depiction of a unit operation useful in the apparatus of FIG. 1 for the selective hydrogenation of carboxylic acid moieties.

FIG. 3 is a schematic depiction of a unit operation useful in the apparatus of FIG. 1 to enhance the mass transfer of hydrogen for the catalytic conversion.

FIG. 4 is a schematic depiction of a unit operation useful in the apparatus of FIG. 1 to rejuvenate heterogeneous, hydrogenation catalyst.

FIG. 5 is a schematic depiction of a unit operation useful in the apparatus of FIG. 1 to recover soluble retro-aldol catalyst, especially tungstate-based retro-aldol catalyst, from a purge stream.

FIG. 6 is a schematic depiction of a unit operation useful in the apparatus of FIG. 1 for the regeneration of retro-aldol catalyst, particularly tungsten-based retro-aldol catalyst, through an oxidation and pH adjustment.

### DETAILED DESCRIPTION

All patents, published patent applications and articles referenced herein are hereby incorporated by reference in their entirety.

### Definitions

As used herein, the following terms have the meanings set forth below unless otherwise stated or clear from the context of their use.

Where ranges are used herein, the end points only of the ranges are stated so as to avoid having to set out at length and describe each and every value included in the range. Any appropriate intermediate value and range between the recited endpoints can be selected. By way of example, if a range of between 0.1 and 1.0 is recited, all intermediate values (e.g., 0.2, 0.3. 0.63, 0.815 and so forth) are included as are all intermediate ranges (e.g., 0.2-0.5, 0.54-0.913, and so forth).

The use of the terms “a” and “an” is intended to include one or more of the element described.

Admixing or admixed means the formation of a physical combination of two or more elements which may have a uniform or non-uniform composition throughout and includes, but is not limited to, solid mixtures, solutions and suspensions.

Aldose means a monosaccharide that contains only a single aldehyde group (CH═O) per molecule and having the generic chemical formula Cn(H2O)n. Non-limiting examples of aldoses include aldohexose (all six-carbon, aldehyde-containing sugars, including glucose, mannose, galactose, allose, altrose, idose, talose, and gulose); aldopentose (all five-carbon aldehyde containing sugars, including xylose, lyxose, ribose, and arabinose); aldotetrose (all four-carbon, aldehyde containing sugars, including erythrose and threose) and aldotriose (all three-carbon aldehyde containing sugars, including glyceraldehyde).

Aldose-yielding carbohydrate means an aldose or a di- or polysaccharide that can yield aldose upon hydrolysis. Sucrose, for example, is an aldose-yielding carbohydrate even though it also yields ketose upon hydrolysis.

Aqueous and aqueous solution mean that water is present but does not require that water be the predominant component. For purposes of illustration and not in limitation, a solution of 90 volume percent of ethylene glycol and 10 volume percent water would be an aqueous solution. Aqueous solutions include liquid media containing dissolved or dispersed components such as, but not in limitation, colloidal suspensions and slurries.

Bio-sourced carbohydrate feedstock means a product that includes carbohydrates sourced, derived or synthesized from, in whole or in significant part, to biological products or renewable agricultural materials (including, but not limited to, plant, animal and marine materials) or forestry materials.

Catalyst for converting the carbohydrate means one or more catalysts to effect the catalytic conversion. For the hydrogenolysis route, catalyst for converting the carbohydrate would include mixtures of hydrogenolysis catalysts as well as a single hydrogenolysis catalyst. For the retro-aldol route, catalyst for converting the carbohydrate included both the retro-aldol catalyst and the hydrogenation catalyst, each of which can comprise one or a mixture of catalysts. The catalyst can contain one or more catalytic metals, and for heterogeneous catalysts, include supports, binders and other adjuvants. Catalytic metals are metals that are in their elemental state or are ionic or covalently bonded. The term catalytic metals refers to metals that are not necessarily in a catalytically active state, but when not in a catalytically active state, have the potential to become catalytically active. Catalytic metals can provide catalytic activity or modify catalytic activity such as promotors, selectivity modifiers, and the like.

Commencing contact means that a fluid starts a contact with a component, e.g., a medium containing a homogeneous or heterogeneous catalyst, but does not require that all molecules of that fluid contact the catalyst.

Compositions of aqueous solutions are determined using gas chromatography for lower boiling components, usually components having 3 or fewer carbons and a normal boiling point less than about 300° C., and high performance liquid chromatography for higher boiling components, usually 3 or more carbons, and those components that are thermally unstable.

Conversion efficiency of aldohexose to ethylene glycol is reported in mass percent and is calculated as the mass of ethylene glycol contained in the product solution divided by the mass of aldohexose theoretically provided by the carbohydrate feed and thus includes any aldohexose per se contained in the carbohydrate feed and the aldohexose theoretically generated upon hydrolysis of any di- or polysaccharide contained in the carbohydrate feed.

Hexitol means a six carbon compound having the empirical formula of C₆H₁₄O₆ with one hydroxyl per carbon.

High shear mixing involves providing a fluid traveling at a different velocity relative to an adjacent area which can be achieved through stationary or moving mechanical means to effect a shear to promote mixing. As used herein, the components being subjected to high shear mixing may be immiscible, partially immiscible or miscible.

Hydraulic distribution means the distribution of an aqueous solution in a vessel including contact with any catalyst contained therein.

Immediately prior to means no intervening unit operation requiring a residence time of more than one minute exists.

Intermittently means from time to time and may be at regular or irregular time intervals.

Ketose means a monosaccharide containing one ketone group per molecule. Non-limiting examples of ketoses include ketohexose (all six-carbon, ketone-containing sugars, including fructose, psicose, sorbose, and tagatose), ketopentose (all five-carbon ketone containing sugars, including xylulose and ribulose), ketotetrose (all four-carbon, ketose containing sugars, including erythrulose), and ketotriose (all three-carbon ketose containing sugars, including dihydroxyacetone).

Liquid medium means the liquid in the reactor. The liquid is a solvent for the carbohydrate, intermediates and products and for the homogeneous, tungsten-containing retro-aldol catalyst. Typically and preferably, the liquid contains at least some water and is thus termed an aqueous medium.

Lower glycol is ethylene glycol or propylene glycol or mixtures thereof.

The pH of an aqueous solution is determined at ambient pressure and temperature. In determining the pH of, for example the aqueous, hydrogenation medium or the product solution, the liquid is cooled and allowed to reside at ambient pressure and temperature for 2 hours before determination of the pH. Where the aqueous solution contains less than about 50 mass percent water, e.g., in a glycol-rich medium, water is added to a sample of the aqueous medium to provide a solution containing about 50 mass percent water. For purposes of consistency, the dilution of solutions is to the same mass percent water.

pH control agents means one or more of buffers and acids or bases.

A pressure sufficient to maintain at least partial hydration of a carbohydrate means that the pressure is sufficient to maintain sufficient water of hydration on the carbohydrate to retard caramelization. At temperatures above the boiling point of water, the pressure is sufficient to enable the water of hydration to be retained on the carbohydrate.

A rapid diffusional mixing is mixing where at least one of the two or more fluids to be mixed is finely divided to facilitate mass transfer to form a substantially uniform composition.

A reactor can be one or more vessels in series or in parallel and a vessel can contain one or more zones. A reactor can be of any suitable design for continuous operation including, but not limited to, tanks and pipe or tubular reactor and can have, if desired, fluid mixing capabilities. Types of reactors include, but are not limited to, laminar flow reactors, fixed bed reactors, slurry reactors, fluidized bed reactors, moving bed reactors, simulated moving bed reactors, trickle-bed reactors, bubble column and loop reactors.

Separation unit operations are one or more operations to selectively separate chemicals, including, but not limited to, chromatographic separation, sorption, membrane separation, flash separation, distillation, rectification, and evaporation.

Soluble means able to form a single liquid phase or to form a colloidal suspension.

Sorption includes absorption (including liquid/liquid extraction), adsorption and ion exchange.

Vapor/liquid separation is a separation providing one or more vapor streams and one or more liquid streams and can be based upon chromatographic separation, cyclic sorption, membrane separation, flash separation, distillation, rectification, and evaporation (e.g., thin film evaporators, falling film evaporators and wiped film evaporators).

### Carbohydrate Feed

The disclosed processes use a carbohydrate feed that contains an aldohexose-yielding carbohydrate or ketose-yielding carbohydrate, the former providing under retro-aldol reaction conditions, an ethylene glycol-rich product and the latter providing a propylene glycol-rich product. Where product solutions containing a high mass ratio of ethylene glycol to propylene glycol are sought, the carbohydrate in the feed comprises at least about 90, preferably at least about 95 or 99, mass percent of aldohexose-yielding carbohydrate. Often the carbohydrate feed comprises a carbohydrate polymer such as starch, cellulose, or partially to essentially fully hydrolyzed fractions of such polymers or mixtures of the polymers or mixtures of the polymers with partially hydrolyzed fractions.

The carbohydrate feed is most often at least one of pentose and hexose or compounds that yield pentose or hexose. Examples of pentose and hexose include xylose, lyxose, ribose, arabinose, xylulose, ribulose, glucose, mannose, galactose, allose, altrose, idose, talose, and gulose fructose, psicose, sorbose, and tagatose. Most bio-sourced carbohydrate feedstocks yield glucose upon being hydrolyzed. Glucose precursors include, but are not limited to, maltose, trehalose, cellobiose, kojibiose, nigerose, nigerose, isomaltose, β,β-trehalose, α,β-trehalose, sophorose, laminaribiose, gentiobiose, and mannobiose. Carbohydrate polymers and oligomers such as hemicellulose, partially hydrolyzed forms of hemicellulose, disaccharides such as sucrose, lactulose, lactose, turanose, maltulose, palatinose, gentiobiulose, melibiose, and melibiulose, or combinations thereof may be used.

The carbohydrate feed can be solid or, preferably, in a liquid suspension or dissolved in a solvent such as water. Where the carbohydrate feed is in a non-aqueous environment, it is preferred that the carbohydrate is at least partially hydrated. Non-aqueous solvents include alkanols, diols and polyols, ethers, or other suitable carbon compounds of 1 to 6 carbon atoms. Solvents include mixed solvents, especially mixed solvents containing water and one of the aforementioned non-aqueous solvents. Certain mixed solvents can have higher concentrations of dissolved hydrogen under the conditions of the hydrogenation reaction and thus reduce the potential for hydrogen starvation. Preferred non-aqueous solvents are those that can be hydrogen donors such as isopropanol. Often these hydrogen donor solvents have the hydroxyl group converted to a carbonyl when donating a hydrogen atom, which carbonyl can be reduced under the conditions in the reaction zone. Most preferably, the carbohydrate feed is provided in an aqueous solution. In any event, the volume of feed and the volume of raw product withdrawn need to balance to provide for a continuous process.

Further considerations in providing the carbohydrate to the reaction zone are minimizing energy and capital costs. For instance, in steady state operation, the solvent contained in the feed exits the reaction zone with the raw products and needs to be separated in order to recover the sought glycol products.

Preferably, the feed is introduced into the reaction zone in a manner such that undue concentrations of HOC's that can result in hydrogen starvation are avoided. With the use of a greater number of multiple locations for the supply of carbohydrate per unit volume of the reaction zone, the more concentrated the carbohydrate in the feed can be. In general, the mass ratio of water to carbohydrate in the carbohydrate feed is preferably in the range of 4:1 to 1:4. Aqueous solutions of 600 or more grams per liter of certain carbohydrates such as dextrose and sucrose are sometimes commercially available.

In some instances, recycled hydrogenation solution having a substantial absence of hydrogenation catalyst, or aliquot or separated portion thereof, is added as a component to the carbohydrate feed. The recycled hydrogenation solution can be one or more of a portion of the raw product stream or an internal recycle where hydrogenation catalyst is removed. Suitable solid separation techniques include, but are not limited to, filtration and density separation such as cyclones, vane separators, and centrifugation. With this recycle, the amount of fresh solvent for the feed is reduced, yet the carbohydrate is fed at a rate sufficient to maintain a high conversion per unit volume of reaction zone. The use of a recycle, especially where the recycle is an aliquot portion of the raw product stream, enables the supply of low concentrations of carbohydrate to the reaction zone while maintaining a high conversion of carbohydrate to ethylene glycol. Additionally, it is feasible to maintain the recycle stream at or near the temperature in the reaction zone and it as it contains tungsten-containing catalyst, retro-aldol conversion can occur prior to entry of the feed into the reaction zone. With the use of recycled hydrogenation solution, the mass ratio of carbohydrate to total recycled product stream and added solvent is often in the range of about 0.05:1 to 0.4:1, and sometimes from about 0.1:1 to 0.3:1. The recycled raw product stream is often from about 20 to 80 volume percent of the product stream.

The carbohydrate contained in the carbohydrate feed is provided at a rate of at least 50 or 100, and preferably, from about 150 to 500 grams per liter of reactor volume per hour. Optionally, a separate reaction zone can be used that contains retro-aldol catalyst with an essential absence of hydrogenation catalyst.

### The Conversion Process

In the processes, the carbohydrate feed is introduced into an aqueous medium that contains catalyst for the catalytic conversion and hydrogen. For the hydrogenolysis route, the catalyst is a hydrogenolysis catalyst, and for the retro-aldol route, a retro-aldol catalyst and hydrogenation catalyst.

### Hydrogenolysis Route

In the hydrogenolysis route, carbon-carbon bonds are cleaved by hydrogen using a hydrogenolysis catalyst under hydrogenolysis conditions. Typically, the carbohydrate feed is contacted with heterogeneous hydrogenolysis catalyst at elevated temperature in the presence of hydrogen to effect the hydrogenolysis and generate ethylene glycol and propylene glycol. The reaction temperatures can fall within a broad range, e.g., from about 120° C. to 300° C., but often temperatures below about 220° C., more particularly below about 200° C., to attenuate the production of 1,2-butanediol. The pressures (absolute) are typically in the range of about 15 to 300 bar (1500 to 30,000 kPa), say, from about 25 to 200 bar (2500 and 20000 kPa). The hydrogen partial pressure is typically in the range of about 15 to 200 bar (1500 to 20,000 kPa), say, from about 25 to 150 bar (2500 and 15000 kPa).

The hydrogenolysis reaction may be carried out in any suitable reactor, including, but not limited to, fixed bed, fluidized bed, trickle bed, moving bed, slurry bed, continuously stirred tank, loop reactors such as Buss Loop® reactors available from BUSS ChemTech AG, and structured bed. The hydrogenolysis catalyst is frequently provided in an amount of from about 0.1 to 10, and more often, from about 0.5 to 5, grams per liter of aqueous medium, and in a packed bed reactor the hydrogenation catalyst comprises about 20 to 80 volume percent of the reactor. The residence time of the aqueous phase in the reactor can vary over a wide range, and is usually from about 1 minute to 5 hours, say, from 5 to 200 minutes. In some instances, the weight hourly space velocity is from about 0.01 to 20 hr-1 based upon total carbohydrate in the feed.

Heterogeneous hydrogenolysis catalysts can be supported and unsupported catalysts. Typical supports include, but are not limited to, silica, zirconia, ceria, titania, alumina, aluminosilicates, clays, carbon such as activated carbon, and magnesia. Hydrogenolysis metals include platinum, palladium, ruthenium, rhodium and iridium, nickel, copper, iron, and cobalt. The hydrogenolysis metals can be used alone or in combination with other hydrogenolysis metals or catalyst modifiers. Rhenium, molybdenum, vanadium, titanium, tungsten, and chromium have been suggested as modifiers. Usually the hydrogenolysis is promoted by base, which is often an alkali metal hydroxide or basic metal oxide. The pH is frequently in the range of about 6 to 12; however, hydrogenolysis can occur at higher and lower acidities.

### Retro-Aldol Route

In the retro-aldol route, the carbohydrate feed may or may not have been subjected to retro-aldol conditions prior to being introduced into the aqueous, hydrogenation medium, and the carbohydrate feed may or may not have been heated through the temperature zone of 170° C. to 230° C. upon contacting the aqueous, hydrogenation medium. Thus, in some instances the retro-aldol reactions may not occur until the carbohydrate feed is introduced into the aqueous medium, and in other instances, the retro-aldol reactions may have at least partially occurred prior to the introduction of the carbohydrate feed into the aqueous, hydrogenation medium. It is generally preferred to quickly disperse the carbohydrate feed in the aqueous, hydrogenation medium especially where the aqueous, hydrogenation medium is used to provide direct heat exchange to the carbohydrate feed. This dispersion can be achieved by any suitable procedure including, but not limited to, the use of mechanical and stationary mixers and rapid diffusional mixing.

The preferred temperatures for retro-aldol reactions are typically from about 230° C. to 300° C., and more preferably from about 240° C. to 280° C., although retro-aldol reactions can occur at lower temperatures, e.g., as low as 90° C. or 150° C. The pressures (absolute) are typically in the range of about 15 to 200 bar (1500 to 20,000 kPa), say, from about 25 to 150 bar (2500 and 15000 kPa). Retro-aldol reaction conditions include the presence of retro-aldol catalyst. A retro-aldol catalyst is a catalyst that catalyzes the retro-aldol reaction. Examples of compounds that can provide retro-aldol catalyst include, but are not limited to, heterogeneous and homogeneous catalysts, including catalyst supported on a carrier, comprising tungsten and its oxides, sulfates, phosphides, nitrides, carbides, halides, acids and the like. Tungsten carbide, soluble phosphotungstens, tungsten oxides supported on zirconia, alumina and alumina-silica are also included. Preferred catalysts are provided by soluble tungsten compounds and mixtures of tungsten compounds. Soluble tungstates include, but are not limited to, ammonium and alkali metal, e.g., sodium and potassium, paratungstate, partially neutralized tungstic acid and ammonium and alkali metal metatungstate. Often the presence of ammonium cation results in the generation of amine by-products that are undesirable in the lower glycol product. Without wishing to be limited to theory, the species that exhibit the catalytic activity may or may not be the same as the soluble tungsten compounds introduced as a catalyst. Rather, a catalytically active species may be formed in the course of the retro-aldol reaction. Tungsten-containing complexes are typically pH dependent. For instance, a solution containing sodium tungstate at a pH greater than 7 will generate sodium metatungstate when the pH is lowered. The form of the complexed tungstate anions is generally pH dependent. The rate that complexed anions formed from the condensation of tungstate anions are formed is influenced by the concentration of tungsten-containing anions. A preferred retro-aldol catalyst comprises ammonium or alkali metal tungstate that becomes partially neutralized with acid, preferably an organic acid of 1 to 6 carbons such as, but without limitation, formic acid, acetic acid, glycolic acid, and lactic acid. The partial neutralization is often from about 25 to 75%, i.e., on average from 25 to 75% of the cations of the tungstate become acid sites. The partial neutralization may occur prior to introducing the tungsten-containing compound into the reactor or with acid contained in the reactor.

The concentration of retro-aldol catalyst used may vary widely and will depend upon the activity of the catalyst and the other conditions of the retro-aldol reaction such as acidity, temperature and concentrations of carbohydrate. Typically, the retro-aldol catalyst is provided in an amount to provide from about 0.05 to 100, say, from about 0.1 to 50, grams of tungsten calculated as the elemental metal per liter of aqueous, hydrogenation medium. The retro-aldol catalyst can be added as a mixture with all or a portion of the carbohydrate feed or as a separate feed to the aqueous, hydrogenation medium or with recycling aqueous medium or any combination thereof. Where the retro-aldol catalyst comprises two or more tungsten species and they may be fed to the reaction zone separately or together.

Frequently the carbohydrate feed is subjected to retro-aldol conditions in a premixing zone prior to being introduced into the aqueous, hydrogenation medium in the reaction zone containing hydrogenation catalyst. Preferably the introduction into the aqueous, hydrogenation medium occurs in less than one minute, and most often less than 10 seconds, from the commencement of subjecting the carbohydrate feed to the retro-aldol conditions. Some, or all of the retro-aldol reaction can occur in the reaction zone containing the hydrogenation catalyst. In any event, the most preferred processes are those having a short duration of time between the retro-aldol conversion and hydrogenation.

The hydrogenation, that is, the addition of hydrogen atoms to an organic compound without cleaving carbon-to-carbon bonds, can be conducted at a temperature in the range of about 100° C. or 120° C. to 300° C. or more. Typically, the aqueous, hydrogenation medium is maintained at a temperature of at least about 230° C. until substantially all carbohydrate is reacted to have the carbohydrate carbon-carbon bonds broken by the retro-aldol reaction, thereby enhancing selectivity to ethylene and propylene glycol. Thereafter, if desired, the temperature of the aqueous, hydrogenation medium can be reduced. However, the hydrogenation proceeds rapidly at these higher temperatures. Thus, the temperatures for hydrogenation reactions are frequently from about 230° C. to 300° C., say, from about 240° C. to 280° C. Typically, in the retro-aldol process the pressures (absolute) are typically in the range of about 15 to 200 bar (1500 to 20,000 kPa), say, from about 25 to 150 bar (2500 to 15000 kPa). The hydrogenation reactions require the presence of hydrogen as well as hydrogenation catalyst. Hydrogen has low solubility in aqueous solutions. The concentration of hydrogen in the aqueous, hydrogenation medium is increased with increased partial pressure of hydrogen in the reaction zone. The pH of the aqueous, hydrogenation medium is often at least about 3, say, from about 3 or 3.5 to 8, and in some instances from about 3.5 or 4 to 7.5.

The hydrogenation is conducted in the presence of a hydrogenation catalyst. Frequently the hydrogenation catalyst is a heterogeneous catalyst. It can be deployed in any suitable manner, including, but not limited to, fixed bed, fluidized bed, trickle bed, moving bed, slurry bed, loop bed such as Buss Loop® reactors available from BUSS ChemTech AG, and structured bed. One type of reactor that can provide high hydrogen concentrations and rapid heating is cavitation reactor such as disclosed in U.S. Pat. No. 8,981,135 B2, herein incorporated by reference in its entirely. Cavitation reactors generate heat in localized regions and thus the temperature in these localized regions rather the bulk temperature of the liquid medium in the reaction zone is the temperature process parameter for purposes of this disclosure. Cavitation reactors are of interest for this process since the retro-aldol conversion can be very rapid at the temperatures that can be achieved in the cavitation reactor.

Nickel, ruthenium, palladium and platinum are among the more widely used reducing metal catalysts. However, many reducing catalysts will work in this application. The reducing catalyst can be chosen from a wide variety of supported transition metal catalysts. Nickel, Pt, Pd and ruthenium as the primary reducing metal components are well known for their ability to reduce carbonyls. One particularly favored catalyst for the reducing catalyst in this process is a supported, Ni—Re catalyst. A similar version of Ni/Re or Ni/Ir can be used with good selectivity for the conversion of the formed glycolaldehyde to ethylene glycol. Nickel-rhenium is a preferred reducing metal catalyst and may be supported on alumina, alumina-silica, silica or other supports. Supported Ni—Re catalysts with B as a promoter are useful. Generally, for slurry reactors, a supported hydrogenation catalyst is provided in an amount of less than 10, and sometimes less than about 5, say, about 0.1 or 0.5 to 3, grams per liter of nickel (calculated as elemental nickel) per liter of liquid medium in the reactor. As stated above, not all the nickel in the catalyst is in the zero-valence state, nor is all the nickel in the zero-valence state readily accessible by glycol aldehyde or hydrogen. Hence, for a particular hydrogenation catalyst, the optimal mass of nickel per liter of liquid medium will vary. For instance, Raney nickel catalysts would provide a much greater concentration of nickel per liter of liquid medium. Frequently in a slurry reactor, the hydrogenation catalyst is provided in an amount of at least about 5 or 10, and more often, from about 10 to 70 or 100, grams per liter of aqueous, hydrogenation medium and in a packed bed reactor the hydrogenation catalyst comprises about 20 to 80 volume percent of the reactor. In some instances, the weight hourly space velocity is from about 0.01 or 0.05 to 1 hr-1 based upon total carbohydrate in the feed. Preferably the residence time is sufficient that glycol aldehyde and glucose are less than 0.1 mass percent of the reaction product, and most preferably are less than 0.001 mass percent of the reaction product.

The carbohydrate feed is at least 50 grams of carbohydrate per liter per hour, and is often in the range of about 100 to 700 or 1000, grams of carbohydrate per liter per hour.

In the disclosed processes, the combination of reaction conditions (e.g., temperature, hydrogen partial pressure, concentration of catalysts, hydraulic distribution, and residence time) are sufficient to convert at least about 95, often at least about 98, mass percent and sometimes essentially all of the carbohydrate that yield aldose or ketose. It is well within the skill of the artisan having the benefit of the disclosure herein to determine the set or sets of conditions that will provide the sought conversion of the carbohydrate.

### DRAWINGS

Reference is made to the drawings which are provided to facilitate the understanding invention but are not intended to be in limitation of the invention. The drawing omits minor equipment such as pumps, compressors, valves, instruments, heat exchangers and other devices the placement of which and operation thereof are well known to those practiced in chemical engineering. The drawing also omits ancillary unit operations.

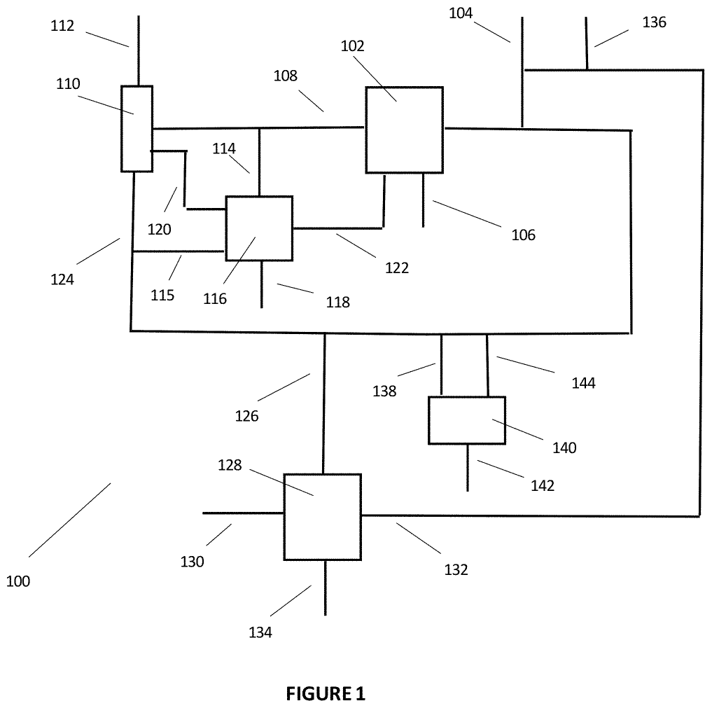

With reference to FIG. 1, apparatus 100 comprises catalytic conversion reactor 102 for the conversion of carbohydrate to ethylene glycol and/or propylene glycol. The conversion can be the hydrogenolysis route or the retro-aldol route. The reactor may be a single vessel or two or more vessels of the same or different design in parallel or in series. Typically, at least one vessel contains heterogeneous hydrogenation catalyst. Where the retro-aldol route is employed, at least one vessel contains retro-aldol catalyst, especially soluble retro-aldol catalyst.

As shown, carbohydrate feed is passed via line 104 to reactor 102, and hydrogen for the catalytic conversion is passed to reactor 102 via line 106. A reaction product is withdrawn from reactor 102 via line 108. The reaction product contains one or both of ethylene glycol and propylene glycol, and it contains by-products and side products such as sorbitol, glycerol, 1,2-butanediol, and the like. Since the catalytic conversion is conducted at elevated pressure in the presence of hydrogen, the reaction product contains dissolved hydrogen. Where the retro-aldol route is used, the reaction product withdrawn from reactor 102 will contain dissolved retro-aldol catalyst. In some instances, the heterogeneous hydrogenation catalyst is also withdrawn from reactor 102 with the reaction product.

In some broad aspects of the processes of this invention, the reaction product is directly passed to vapor/liquid separator 110. The vapor/liquid separator may comprise one or more unit operations, e.g., with recovery of hydrogen and light gases followed by one or more unit operations to recover water and ethylene glycol and propylene glycol from the aqueous medium. For the sake of convenience, the drawing indicates only one vapor discharge line 112. The vapor/liquid separator can be operated in any convenient mode.

In one mode, normally gaseous components in the reaction product, for instance, hydrogen, methane, carbon monoxide, and carbon dioxide are separated and discharged via line 112 for waste or recovery. The liquid components can then be subjected to one or more unit operations to recover lower glycol including additional vapor/liquid separations or liquid/liquid separations such as selective membrane permeation and selective sorption. In another mode, the vapor/liquid separation provides a vaporous overhead that contains a substantial portion of the ethylene glycol and propylene glycol in the reaction product. Often, at least about 30, and more frequently at least about 50, say, about 50 to 75 or 95, mass percent of the total ethylene glycol and propylene glycol are provided to the overhead. The overhead in line 112 would be passed to unit operations for the refining of ethylene glycol and propylene glycol as well as separation of normally gaseous components.

In some aspects, a unit operation can be interposed between reactor 102 and vapor/liquid separator 110. For instance, all or a portion of the reaction product in line 108 can be passed via line 114 to unit operation 116. Unit operation 116 can comprise one or more unit operations. Line 118 supplies material to the unit operation 116. And line 120 is adapted to direct material in unit operation 116 to another part of the process, and as depicted, but not in limitation, to vapor/liquid separator 110. Line 122 is adapted to pass all or a portion of the material in unit operation to reactor 102.

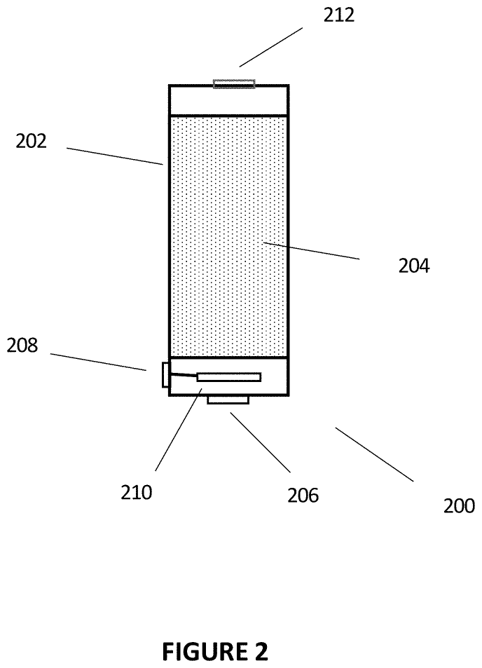

FIG. 2 depicts one type of unit operation 116, which is a hydrogenation reactor assembly 200 adapted to selectively convert carboxylic moieties contained in the reaction product to alcohols thereby reducing the acid content of the reactor product often by at least about 50, and often at least about 75, mass percent. Hydrogenation reactor assembly 200 comprises hydrogenation reactor 202 having heterogeneous catalyst 204 therein. Reactor 202 may be of any suitable configuration such as a packed bed, ebulating bed, fluidized bed, moving bed, continuously stirred bed, loop reactor and moving bed reactor. The hydrogenation catalyst is preferably a heterogeneous catalyst and the catalyst may be supported or unsupported. The hydrogenation catalyst is selective for the hydrogenation of carboxylic acids as compared to hydroxyl groups. Hydrogenation catalysts that have been proposed for selective hydrogenation of acids include, but are not limited to, catalysts containing one or more of cobalt, copper, ruthenium, platinum and palladium, and the catalysts may include one or more other metals such as tin and rhenium. Supports include aluminas, silicas, aluminosilicates, activated carbon and the like.

Reactor 202 is provided with inlet port 206 which is adapted to receive reaction product from line 115 of FIG. 1. All or a portion of the reaction product withdrawn from reactor 102 can be passed to reactor 202. Reactor 202 is provided with hydrogen inlet port 208 which is adapted to receive hydrogen for the hydrogenation via line 118 of FIG. 1. The hydrogen that is supplied is shown as being distributed by distributor 210 and admixed with the reaction product prior to contacting hydrogenation catalyst 204. Outlet port 212 is provided on reactor 202 to withdrawn hydrogenated reaction product from reactor 202. Port 212 is adapted to supply the hydrogenated reaction product to one or both of lines 120 and 122 of FIG. 1. In most instances, substantially all of the hydrogenated reaction product is recycled to reactor 102. As shown in FIG. 1, the recycle may be directly to reactor 102. However, it is to be understood that the recycling, hydrogenated reaction product can be admixed with other feeds to reactor 102, e.g., with the carbohydrate feed, with replenishment catalyst, or with adjuvants.

The selective hydrogenation conditions are well known to those skilled in the art and can be optimized for the sought degree of reduction of carboxylic acid in the reaction product. Typically, the hydrogenation temperatures are from about 120° C. to 300° C. and the hydrogen partial pressure is from about 2000 to 20,000, say, about 2500 to 10,000, kPa. The liquid hourly space velocity, which is the volume of reaction product per volume of hydrogenation catalyst per hour, is sometimes in the range of about 0.5 to 10. It is to be understood that the optimal hydrogenation conditions for the selective hydrogenation of the acid groups will depend, in part, upon the type of catalyst used.

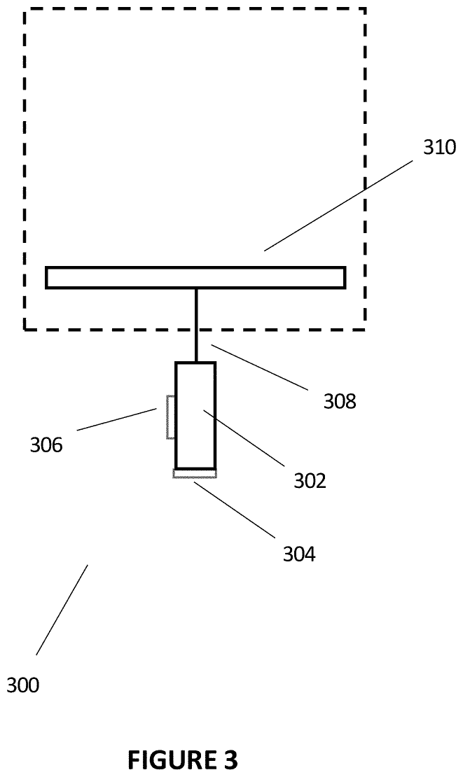

In another embodiment unit operation 116 serves to assist in introducing and distributing hydrogen in reactor 102. Turning to FIG. 3, hydrogen distribution unit operation 300 is used to introduce hydrogen into reactor 102 via a motive fluid. Venturi mixer 302 is provided with liquid feed port 304 which is adapted to be connected to line 114 of FIG. 1. Hydrogen is introduced into venture mixer 302 via port 304 which is adapted to be connected to line 118 of FIG. 1. Hydrogen is admixed with reaction product in venture mixer 302 and the gas and liquid mixture is then passed via line 308 to injector 310 which is located in reactor 102. Injector 310 may be jet mixers/aerators or slot injectors. Slot injectors are preferred, one form of which is disclosed in U.S. Pat. No. 4,162,970. These injectors operate using a motive liquid, which is conveniently the reaction product withdrawn from reactor 102. The injectors, especially slot injectors, are capable of operating over a wide range of liquid and gas flow rates and thus are capable of significant turn down in gas transfer capability. The injectors are characterized as having nozzles of at least about 1, often about 1.5 to 5, say, 2 to 4, centimeters as the cross-sectional dimension in the case of jet injectors or as the smaller cross-sectional dimension in the case of slot injectors. The energy required to provide microbubbles of a given size is often less than that required to form microbubbles of that size using a microbubble sparger. The bubble size generated by the injectors will be influenced by, among other factors, the rate of liquid flow through the injector and the ratio of hydrogen to reaction product passing through the injector. Preferably the hydrogen introduced by the injector is in the form of microbubbles having diameters in the range of 0.01 to 0.5, preferably 0.02 to 0.3 millimeter. The microbubbles serve to enhance the rate of mass transfer of hydrogen to the medium in rector 102.

The flow rate of reaction product used in an injector will depend upon the type, size and configuration of the injector and the sought bubble size of the gas feed. In general, the velocity of the dispersion stream leaving the injector is frequently in the range of 0.5 to 5 meters per second and the ratio of hydrogen to motive liquid is in the range of from about 1:1 to 3:1 actual cubic meters per cubic meter of motive liquid.

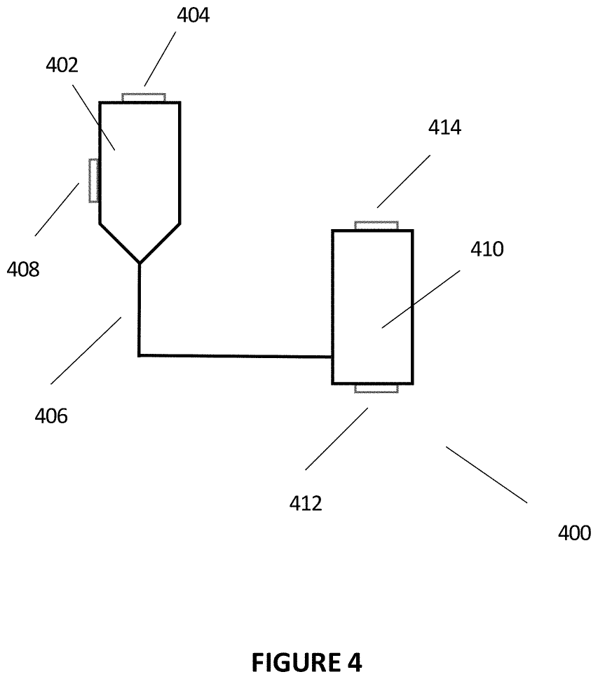

In FIG. 4, unit operation 116 comprises a heterogeneous catalyst rejuvenation system generally designated by the numeral 400. System 400 comprises a solids concentrator 402 and catalyst hydrotreater 410. Solids concentrator 400 may be any suitable unit operation to provide a solids lean stream and a solids rich stream. Examples of solids concentrators are filters, hydrocyclones, vane separators, settling tanks, and centrifuges. A reaction product is withdrawn from reactor 102. The reaction product contains heterogeneous catalyst. At least a portion of the withdrawn reaction product is passed continuously or intermittently as needed to heterogeneous catalyst rejuvenation system 400. Solids concentrator 402 is depicted as having port 404 adapted to connect to line 114 of FIG. 1 and supply reaction product containing heterogeneous catalyst to solids concentrator 402. In solids concentrator 402, a solids rich stream exits via line 406 and a solids lean stream exits via port 408. Port 408 is adapted to be connected to line 120 to be passed to vapor/liquid separator 110. In some embodiments, no solids concentrator 402 is used.

The solids rich stream is passed via line 406 to hydrotreater 410. Hydrotreater 410 can be a vessel providing a residence time sufficient to reduce at least a portion of oxidized metal contained in the hydrogenation catalyst. Hydrotreater 410 has a hydrogen port 412 adapted to connect to line 118 of FIG. 1 for the supply of hydrogen, and exit port 414 adapted to be connected to line 122 of FIG. 1 for recycling the reactor product with treated heterogeneous catalyst to reactor 102 of FIG. 1.

As stated above, reaction product with heterogeneous catalyst can be continuously or intermittently withdrawn from reactor 102 for rejuvenation. Although the catalytic conversion of carbohydrate to ethylene glycol and propylene glycol is conducted under reducing conditions, the presence of oxygenated moieties, especially in regions where the catalyst may be hydrogen starved, can result in some oxidation of catalytic metals and thus loss of hydrogenation activity. Rejuvenation can enhance the activity of the hydrogenation catalyst. The rejuvenation may thus be conducted only when a loss of hydrogenation activity is observed; however, continuous or more frequent, intermittent operations can be used to attenuate the risk of loss of catalytic activity. The rejuvenation unit operation can also serve to saturate with hydrogen the catalyst and recycling liquid as a means to supply hydrogen to reactor 102. Typically, the rejuvenation by hydrogen is for a duration of from about 1 minute to 10 hours, say, from about 5 to 200 minutes. The temperature of the rejuvenation is often in the range of about 150° C. to 400° C. or more, and the hydrogen partial pressure is in the range of about 2000 to 20,000, e.g., 3000 to 15,000, kPa. Other techniques can be used to facilitate the rejuvenation or activation of the hydrogenation catalyst or hydrogenolysis catalyst alone or in combination with reducing with hydrogen. For instance, the catalyst can be treated with hydrazine or borohydride or subjected to oxidation, e.g., with oxygen or peroxide, before reduction.

Returning to FIG. 1, all, none or a portion (aliquot or aliquant) of the reaction product withdrawn from reactor 102 is passed via line 108 to vapor/liquid separator 110. Vapor/liquid separator 110 can comprise one or more unit operations. The unit operations used in separator 110 may be of any suitable type, including, but not limited to, chromatic separation such as simulated moving bed chromatography, cyclic sorption, a membrane separator, a flash separator, distillation column, and evaporators such as thin film evaporators, falling film evaporators and wiped film evaporators. The vapor/liquid separator can comprise one or more unit operations in parallel or in series, and provide one or more vaporous streams and one or more liquid streams. It is to be understood that more than line 112 can exist, with different vapor compositions in each. As depicted, a portion of the ethylene glycol and propylene glycol passed to separator 110 is recovered in the vapor phase. The one or more vapor phases that exit vapor/liquid separator 110 via one or more lines 112 can be subjected to further separation and refining. Often, hydrogen and light gases such as methane, carbon monoxide and carbon dioxide are flashed in a first unit operation of vapor/liquid separator 110 and either used for heat generation or subjected to separation processes to recover and recycle hydrogen. Low boiling components in the condensables in the vapor phase from one or more lines 112 from one or more unit operations comprising vapor/liquid separator 110, such as ethanol and methanol, can be recovered via distillation. Water can also be recovered via distillation followed by recovery of propylene glycol and ethylene glycol and removal of co-products such as 1,2-butanediol. It is typical in the manufacture of ethylene glycol to use multi-effected evaporators to minimize energy usage in the recovery of the ethylene glycol. In some instances, separation of the ethylene glycol from the propylene glycol or other close boiling glycols is effected by an additional, more sophisticated separation technology. Simulated moving bed technology is one such option that can be used. The choice is dependent, in part, on the quality of the product that is required by the desired end use for the product and energy consumption and balance in the plant. At least one liquid phase, or bottoms stream, is provided by the vapor/liquid separator 110 and exits via line 124.

Often vapor/liquid separator 110 comprises a vapor/liquid separation unit operation conducted at lower pressures and temperatures than those in reactor 102. Where a distillation, flash or evaporation, the bottoms temperature is frequently in the range of about 120° C. to 200° C., and the vapor phase is at a pressure of from about 500 to 10,000, say, 1000 to 5000, kPa absolute.

As most of the water and total ethylene glycol and propylene glycol are passed to the vapor phase in the preferred embodiments, the liquid phase may sometimes be rich in heavies and thus increase the difficulties in processing. Accordingly, water is preferably added to the liquid phase from the vapor/liquid separator to provide a liquid comprising at least about 25, and sometimes at least about 35, mass percent water.

All or a portion (aliquot or aliquant) of the liquid phase from vapor/liquid separator 110 can be recycled to reactor 102 via line 124. The liquid phase that is recycled can optionally be heated to assist in maintaining the aqueous medium in reactor 102 at the sought temperature for the catalytic conversion. One or more components being supplied to reactor 102 can, if desired, be admixed with the recycling liquid phase prior to introduction into reactor 102. Such components include, but are not limited to hydrogen, carbohydrate feed, catalyst, pH modifiers, and adjuvants. Where the catalytic conversion is by the retro-aldol route, admixing carbohydrate feed and retro-aldol catalyst is sometimes a preferred mode of operation. See, for instance, U.S. published patent applications 2017/0349513 and 2018/0086681 and U.S. Pat. Nos. 9,399,610 and 9,783,472, all hereby incorporated by reference in their entireties. In some instances, the admixing of a heated liquid phase with carbohydrate feed can facilitate a rapid heating of the carbohydrate through a temperature zone of 170° C. to 230° C. which in some instances reduce isomerization of the carbohydrate.

Where a homogeneous, retro-aldol catalyst is used for the catalytic conversion, the liquid phase from vapor/liquid separator 110 will typically contain substantially all of the retro-aldol catalyst and other compounds and complexes derived therefrom in the reaction product supplied to it. The recycle of the liquid phase thus serves to conserve the retro-aldol catalyst. Similarly, any particulate heterogeneous catalyst would also be conserved due to the recycle of the liquid phase.

Especially where the retro-aldol route is being used, the presence of hydrogenation catalyst in the recycled liquid phase that becomes admixed with carbohydrate feed can be a consideration as any hydrogenolysis of the carbohydrate feed can reduce selectivities to ethylene glycol and propylene glycol. In one preferred embodiment, the partial pressure of hydrogen in the liquid phase is such that when the liquid phase is contacted with carbohydrate, substantially no hydrogenolysis would occur. Often, the partial pressure of hydrogen in the liquid phase from the vapor/liquid separator is less than about 1000, preferably less than 500, kilopascal, until the liquid phase is passed to the reaction zone.

Where the catalytic conversion is via hydrogenolysis in the presence of a hydrogenolysis catalyst, and the liquid phase from vapor/liquid separator 110 contains heterogeneous hydrogenolysis catalyst, the occurrence of hydrogenolysis prior to introduction of the liquid phase into reactor 102 can occur and may, in some instances, be desired. In the latter case, the liquid phase as it is being passed to the reaction zone is under hydrogenolysis conditions and at least a portion of the carbohydrate is catalytically converted to ethylene glycol and propylene glycol. The hydrogenolysis conditions often include a hydrogen partial pressure of at least about 2000, say, from 3000 to 20,000, kPa and temperatures above about 150° C. Hydrogen may be introduced into the liquid phase prior to entry into reactor 102 to facilitate hydrogenolysis of the carbohydrate feed admixed with the liquid phase.

The catalytic conversion process disclosed herein can be conducted on a continuous basis for a duration, and then the process stopped for a turn around. The operator could determine the duration of on-line time based upon performance such as selectivity and conversion rate, catalyst aging or loss, and the build-up of undesired coproducts and by-products or other materials. Often a portion of the liquid phase being recycled to reactor 102 is continuously or intermittently withdrawn as a purge via line 126 to prevent undesired build-up of coproducts and by-products or other materials. In instances where a gas phase is recycled, e.g., hydrogen-containing gas, it is possible that gas phase inerts such as methane, carbon dioxide, nitrogen, etc., build up. In those instances, a gas-phase purge can be continuously or intermittently effected.

The frequency and amounts of the purge can be the same or vary over the duration of the operation of the apparatus. Often the frequency and amounts of the purge reflect the performance of the process and composition of the liquid phase at any given time.

For example, the buildup of inerts (including substantially inert compounds) in the liquid phase, and thus reactor 102, can be a determinant for the frequency and amount of the purge. Inerts include higher molecular weight carboxylic acids and alcohols that are not removed in vapor/liquid separator 110. Examples of substantially inert compounds that are coproducts of the catalytic conversion include sorbitol, glycerol, erythitol and threitol, with sorbitol and glycerol being the most prevalent. In some instances, the conditions for the catalytic conversion are sufficient to convert glycerol to propylene glycol and sorbitol to ethylene glycol and propylene glycol. In these instances, the concentration of sorbitol is sometimes allowed to buildup to at least about 3, say, at least about 5, and sometimes from about 5 to 20, mass percent of the reaction product from reactor 102. The purge can also remove solids, e.g., from the degradation of catalysts, solids in the purge, or solids formed by precipitation.

In another embodiment, the purge rate is sufficient to maintain the pH of the aqueous medium withdrawn from the reaction zone before it is subjected to the vapor/liquid separation, within a sought range, say, within a pH range of +/−2, and preferably +/−1.5 pH units, of the targeted range. For the hydrogenolysis route, the targeted pH often is in the range of about 3 to 12, and sometimes at the more acidic or more basic portions of that range are preferred, and for the retro-aldol route, in the range of about 3 to 8, frequently about 3 or 3.5 to 8, say, 3.5 or 4 to 6.5.

In yet another embodiment, at least one catalyst or catalyst component for the catalytic conversion degrades or becomes inactive. For instance, retro-aldol catalysts such as those based upon tungstate can convert to inactive tungsten species, and components of heterogeneous catalysts such as hydrogenation metals, promoters and supports can dissolve or form particles that may, or may not, have catalytic activity. The frequency and amount of purge can be sufficient to prevent an undesirable buildup of these components. Alternatively, the purge can be used to provide a stream from which one or more of these components can be recovered.

Where particulate solids are generated in the catalytic conversion, the purge rate is preferably sufficient to maintain the concentration of particulate solids in the withdrawn aqueous medium from the reaction zone substantially constant. By substantially constant, the concentration can vary within a range of from about +/−20, to preferably +/−10, percentage points. The particulate solids can include fragmented and precipitated solids derived from the catalysts or supports, and for the retro-aldol route, from the homogeneous retro aldol catalyst. In some instances, conditions in the reaction zone can affect the formation of particulates, and hence, the purge rate can vary over time.

In accordance with an embodiment, where the purge contains components from at least one catalyst used in the catalytic conversion, the purge is subjected to one or more unit operations to recover catalytic metals or compounds from the purge.

As depicted in FIG. 1, the purge in line 126 is passed to one or more unit operations 128 to recover components of one or more catalysts used for the catalytic conversion. Line 130 can be one or more conduits to provide materials for recovering the components in unit operation 128, and line 132 is an optional line to return active catalyst to reactor 102. Line 134 serves to discharge remaining purge from unit operation 128. Unit operation 128 can be one or more of filtration; ion exchange, including anionic and cationic exchange; settling; centrifugation; and magnetic separation. Unit operation 128 can include operations to cause dissolved or small particulates to precipitated or agglomerate to sizes that can facilitate separation. The recovered components can be recovered or, in some instances, can be converted to a form that can be recycled to reactor 102 to provide catalytic activity.

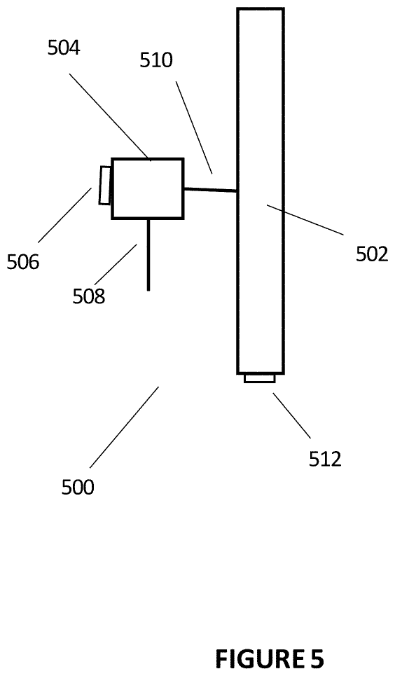

FIG. 5 depicts unit operation 128 that is a filtration apparatus generally designated as 500. A bank of ultrafiltration membranes 502 is used to separate precipitated and particulate catalyst components from the purge. Often, the filtration media have an effective pore size of less than about 100, preferably less than about 50, and sometimes less than about 10, nanometers. As shown, a precipitator 504 is used to convert soluble retro-aldol catalyst and soluble species from the retro-aldol catalyst to solids for separation by membranes 502. Port 506 is provided on precipitator 504 and is adapted to be in fluid flow connection to line 126 of FIG. 1. An aqueous acidic solution is provided to precipitator 505 via line 508. The acid addition is preferably sufficient to lower the pH to less than about 3.5, more preferably less than 3. The acidic solution may be any aqueous acidic solution, e.g., a mineral acid solution such as of hydrochloric, sulfuric or sulfonic acid. At the low pH, the soluble retro-aldol catalyst components are converted to acids that precipitate. For instance, tungstic acid is relatively insoluble in water. Alternatively, line 508 can supply cations that results in the precipitation of anionic components derived from the catalysts. The cations can be monovalent, divalent or polyvalent, for example, silver, magnesium, calcium, iron or copper. The temperature for the precipitation can vary over a wide range and is usually from about 5° C. to 150° C., and the residence time in precipitator 504 is often from 1 minute to 10 hours; however, with some cations, especially in the presence of lower glycol, the rate that solids of a size facilitating recovery are formed, can be longer. Alternatively, an activated carbon bed can be used to recover homogeneous retro-aldol catalyst, for instance, tungsten-containing catalyst.

The purge containing the precipitated materials is passed via line 510 to membranes 502. The solids lean purge exits membranes 502 via port 512 which is adapted to be in fluid communication with line 134 of FIG. 1. Catalytic components can be recovered from the spent membranes as is well known in the art. In one recovery operation, where the membrane has retained precipitates from the retro-aldol catalyst, membrane 502 can be taken off stream and an aqueous basic solution can be passed through membrane 502 to redissolve the retro-aldol catalyst by converting it to a soluble salt. The soluble retro-aldol catalyst salt can be passed via line 132 of FIG. 1 to reactor 102. Often the pH is increased to achieve a pH from about 3.5 to 8, say 4 to 7.

In another mode where the retro-aldol route is being used with a soluble tungsten-containing catalyst, and reduced tungsten-containing species, which may be solid or ionized form in the reaction zone, the reduced tungsten-containing species can be converted to soluble tungstate species. Any suitable oxidant can be used such as oxygen, ozone, peroxides, e.g., hydrogen peroxide, hydroperoxides, peroxyacids, diacyl peroxides, dialkyl peroxides, such as peracetic acid, and soluble peracid and peroxyanion compounds such as peroxycarbonate, perchlorate and permanganate.

As shown for the retro-aldol route, supplemental retro-aldol catalyst can be supplied to reactor 102 via line 136. It should be understood that retro-aldol catalyst may be recycled via at least one of line 122 and line 124, and the supplemental retro-aldol catalyst can be introduced into either or both of these lines or directly into reactor 102 or combined with feed prior to being introduced into reactor 102. The supplemental supply can be continuous or intermittent, and the amount supplied can vary over the duration of the catalytic conversion run. In one mode of operation, the retro-aldol route is used and the carbohydrate feed is admixed with retro-aldol catalyst prior to being introduced into reactor 102. Hence, some retro-aldol reaction can occur prior to the introduction of the feed into reactor 102. In this mode, one preferred embodiment is to control the rate of supply of supplemental retro-aldol catalyst to provide optimal conversion of the carbohydrate to ethylene glycol and propylene glycol as compared to sorbitol and 1,2-butanediol.

Returning to FIG. 1, line 124 recycles liquid phase from vapor/liquid separator 110 to reactor 102. An in-line unit operation system 140 is adapted to treat all or a portion of the liquid phase. Line 138 continuously or intermittently supplies liquid phase to unit operation system 140 for treatment. Line 144 returns treated liquid phase to line 124. Line 142 can either supply material for treating the liquid phase or to remove material generated by the treatment. Whether all or a portion of the liquid phase is passed to unit operation system 140, and whether the supply of liquid phase is continuous or intermittent and the duration of supply if intermittent, depends upon the type of unit operation and the objective of the operator.

In one embodiment, hydrogen is supplied via line 142 to unit operation system 140 to provide a portion of the hydrogen passing to reactor 102. Often the hydrogen supplied provides a partial pressure in the recycling liquid phase of from about 2000 to 50,000, often from about 4000 to 25,000, kilopascals. By supplying hydrogen with the recycle, hydrogen mass transfer and distribution within reactor 102 can be enhanced. In some instances, the recycling liquid phase can be used as the motive fluid for injectors, or eductors, to introduce small bubbles of hydrogen in the aqueous medium in reactor 102. Where the recycling liquid phase contains hydrogenation or hydrogenolysis catalyst, the duration of contact and conditions of temperature and pressure can result in the surface of the catalytic metal or metals to become laden with hydrogen and in some instances can be sufficient to reduce metal of the hydrogenation or hydrogenolysis catalyst.

Unit operation system 140 can comprise unit operations for the separation of dissolved or particulate metals, e.g., from the catalysts and supports. Where such metals are dissolved, the removal can be by any suitable unit operation such as membrane separation, magnetic separation, ion exchange and chemical reaction to precipitate such dissolved metals. In instances where such metals are contained in particles, the removal can be by any suitable unit operation such as filtration and density separation. Examples of density separation include, but are not limited to, gravity settling, cyclonic and centrifugation. In some instances, separations are enhanced by the addition of coagulants or flocculants such as polymeric agents although inorganic agents such as alum can be used but it is preferred that the aqueous medium returning to the reaction zone be substantially free of such coagulants or flocculants. Reference is made to the discussion pertaining to unit operation 128 as the same general techniques and procedures can be used.

In another embodiment, unit operation system 140 comprises a selective catalytic hydrogenation to convert carboxylic acid to alcohol. Similar to that described in connection with unit operation 116, the liquid phase is subjected to carboxylic acid hydrogenation conditions including the presence of a carboxylic acid hydrogenation catalyst and hydrogen at elevated temperature and pressure. Non-limiting examples of carboxylic acid reducing catalytic metals are copper, platinum and ruthenium. Preferably the carboxylic acid reducing catalyst is supported to facilitate separation from the aqueous medium. Supports include, but are not limited to, activated carbon, silica, silica alumina, alumina such as gamma, transition aluminas and alpha alumina, zirconia, titania, and ceria. Acid hydrogenation conditions include temperatures of from about 150° C. to 300° C. and hydrogen partial pressures of from about 2000 to 50,000, often from about 4000 to 25,000, kilopascals.

Unit operation system can alternatively be a unit operation for separating at least a portion of the organic acids from the liquid phase using unit operations known in the art such as, but not in limitation, sorption and simulated moving bed chromatography. The recovered acids may find commercial value.

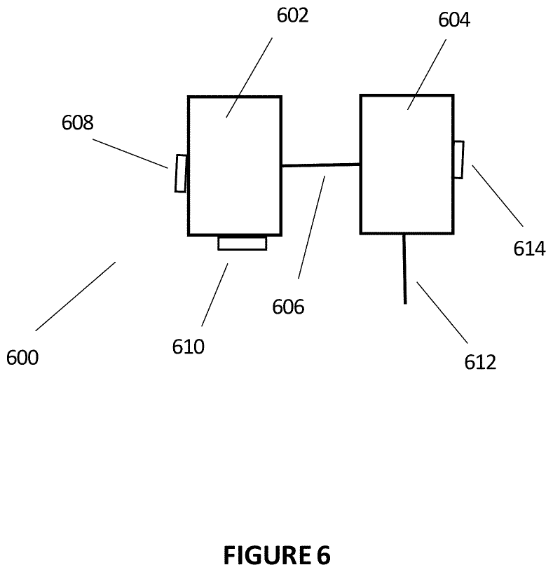

FIG. 6 depicts unit operation 600 that can be used in unit operation system 140. In this embodiment, a retro-aldol route is being used for the catalytic conversion and a soluble tungsten-containing catalyst is the retro-aldol catalyst. In use, solid tungstate species can form in the aqueous medium in rector 102. These solids are contained in the liquid phase being recycled to reactor 102 via line 124. Since the liquid phase has been subjected to a vapor/liquid separation, the dissolved hydrogen concentration can be very low, e.g., less than about 5, and sometimes less than about 1, milligram per kilogram of liquid phase. All or a portion of the liquid phase is passed via line 138 from line 124 to port 608 of oxidizer 602. In oxidizer 602 the liquid phase is subjected to an oxidation unit operation to convert solid tungsten species to soluble tungstate species. The oxidant for the oxidation is provided via line 134 of FIG. 1 to port 610 of oxidizer 602. Any suitable oxidant can be used such as oxygen, ozone, peroxides, e.g., hydrogen peroxide, hydroperoxides, peroxyacids, diacyl peroxides, dialkyl peroxides, such as peracetic acid, and soluble peracid and peroxyanion compounds such as peroxycarbonate, perchlorate and permanganate. Oxidizer 602 may be of any convenient design and preferably provides for static mixing or mechanical mixing. The temperature of the oxidation is usually within the range of about 15° C. to 200° C., say, 25° C. to 125° C., although higher or lower temperatures can be operable. The contact time with the oxidant is generally below about 24 hours, say, 5 seconds to 10 hours. The amount of oxidant provided is frequently less than about 1 gram per liter of liquid phase, say, from about 0.01 to 0.5, gram per liter of liquid phase.

As depicted, the oxidant treated liquid phase from oxidizer 602 is passed via line 606 to pH conditioner 604. In conditioner 604, base or buffer is provided via line 612 and serves to adjust the pH of the liquid phase to from about 3.5 to 8, preferably, 4 to 6.5 or 7, where tungstate species are formed that have desirable retro-aldol activity. The preferred base is alkali metal hydroxide, especially sodium hydroxide, and a preferred pH control agent is sodium tungstate. Tungstate chemistry is complex and various species can exist. By adjusting the pH to a sought level, the concentration of catalytically-active species can be optimized. Port 614 of pH conditioner 604 is adapted to be in fluid communication with line 144 of FIG. 1 for recycle to reactor 102.

Although the disclosure has been described with references to various embodiments, persons skilled in the art will recognized that changes may be made in form and detail without departing from the spirit and scope of this disclosure.

What is claimed is:

1. A catalytic process for producing a lower glycol of at least one of ethylene glycol and propylene glycol from a carbohydrate-containing feed comprising at least one of aldose- and ketose-yielding carbohydrate, said process comprising continuously or intermittently supplying the feed to a reaction zone containing an aqueous medium having therein one or more catalysts for converting said carbohydrate to said glycol, wherein at least one of the catalysts is dissolved or suspended in the aqueous medium, said aqueous medium being at catalytic conversion conditions including the presence of dissolved hydrogen, to produce a reaction product containing said lower glycol, wherein
(i) continuously or intermittently at least a portion of the aqueous medium containing said dissolved or suspended catalyst is withdrawn from the reaction zone;
(ii) at least a portion of the withdrawn aqueous medium is subjected to one or more unit operations to remove a portion of the lower glycol in a separated fraction and provide a retained liquid phase containing at least about 10 mass percent of the lower glycol that was contained in the aqueous medium as withdrawn from the reaction zone and said dissolved or suspended catalyst; and
(iii) at least a portion of the liquid phase containing the dissolved or suspended catalyst from the one or more unit operations is passed to the reaction zone.

2. The process of claim 1 wherein the reaction product contains organic acid, and at least about 25 mass percent of the organic acid is, in the one or more unit operations to remove a portion of the lower glycol, separated to a separated fraction.

3. The process of claim 1 wherein the one or more unit operations is a vapor/liquid separator and wherein water is added to the liquid phase from the vapor/liquid separator to provide a liquid phase comprising at least about 25 mass percent water.

4. The process of claim 1 wherein the mass ratio of lower glycol to water in the retained liquid phase is at least about 20:1.

5. The process of claim 1 wherein the catalyst for the catalytic process comprises a homogeneous retro-aldol catalyst and a heterogeneous hydrogenation catalyst, and the retained liquid phase contains the retro-aldol catalyst and at least a portion of the retro-aldol catalyst is passed with the liquid phase to the reaction zone.

6. The process of claim 1 wherein at least a portion of the retained liquid phase is continuously or intermittently removed as a liquid phase purge.

7. The process of claim 1 wherein the one or more unit operations to remove lower glycol from the withdrawn aqueous medium is a vapor/liquid separation.

8. A catalytic process for producing a lower glycol of at least one of ethylene glycol and propylene glycol from a carbohydrate-containing feed comprising at least one of aldose- and ketose-yielding carbohydrate, said process comprising continuously or intermittently supplying the feed to a reaction zone containing an aqueous medium having therein one or more catalysts for converting said carbohydrate to said glycol, said aqueous medium being at catalytic conversion conditions including the presence of dissolved hydrogen, to produce a reaction product containing said lower glycol and organic acid, wherein
(i) continuously or intermittently at least a portion of the aqueous medium is withdrawn from the reaction zone;
(ii) at least a portion of the withdrawn aqueous medium is subjected to at least one unit operation sufficient to remove at least about 25 mass percent of the organic acid contained in the withdrawn aqueous medium and provide a retained liquid phase; and
(iii) at least a portion of the liquid phase is passed to the reaction zone.

9. The process of claim 8 wherein the one unit operation comprises a vapor/liquid separation providing a vapor phase that removes a portion of the lower glycol to the vapor phase and at least about 35 mass percent of the organic acid is separated to the vapor phase.

10. A catalytic process for producing a lower glycol of at least one of ethylene glycol and propylene glycol from a carbohydrate-containing feed comprising at least one of aldose- and ketose-yielding carbohydrate, said process comprising continuously or intermittently supplying the feed to a reaction zone containing an aqueous medium having therein one or more catalysts for converting said carbohydrate to said glycol, said aqueous medium being at catalytic conversion conditions including the presence of dissolved hydrogen, to produce a reaction product containing said lower glycol and wherein solids are generated, wherein
(i) continuously or intermittently at least a portion of the aqueous medium is withdrawn from the reaction zone;
(ii) at least a portion of the withdrawn aqueous medium is subjected to one or more separation unit operations, preferably comprising a vapor/liquid separation, to remove at least a portion of the lower glycol and provide a remaining liquid phase;
(iii) a portion of the remaining liquid phase is passed to the reaction zone, and
(iv) continuously or intermittently a portion of the liquid phase is purged to maintain the mass of solids per unit volume of the aqueous medium substantially constant.

11. The process of claim 10 wherein the purge is subjected to one or more unit operations to recover catalytic metals from the purge.

12. The process of claim 11 wherein catalytic metals are components of the heterogeneous catalyst

13. The process of claim 11 wherein retro-aldol catalyst is used and retro-aldol catalyst is recovered from the purge.

14. The process of claim 11 wherein the catalytic metals are metal-containing ions and are recovered by ion exchange or through chemical treatment by at least one of:
(i) introducing counter ions to precipitate and
(ii) causing an oxidation or reduction of the metal-containing ions into solid form.

15. A catalytic process for producing a lower glycol of at least one of ethylene glycol and propylene glycol from a carbohydrate-containing feed comprising at least one of aldose- and ketose-yielding carbohydrate, said process comprising continuously or intermittently supplying the feed to a reaction zone containing an aqueous medium having therein catalyst for converting said carbohydrate to said glycol, said aqueous medium being at catalytic conversion conditions including the presence of dissolved hydrogen, to produce a reaction product containing said glycol, wherein
(i) continuously or intermittently at least a portion of the aqueous medium is withdrawn from the reaction zone, and
(ii) at least a portion of the withdrawn aqueous medium from the reaction zone is recycled to the reaction zone,
wherein in step (ii) the aqueous medium recycling to the reaction zone is subjected to at least one unit operation to enhance the catalytic process in the reaction zone.

16. The process of claim 15 wherein the aqueous medium withdrawn from the reaction zone comprises catalytic metals and the at least one unit operation to enhance the catalytic process comprises subjecting the aqueous medium to a unit operation that selectively reduces concentration of catalytic metals.

17. The process of claim 16 wherein the unit operation is a density separation.

18. The process of claim 16 wherein the catalyst for converting the carbohydrate to glycol comprises retro-aldol catalyst and the retro-aldol catalyst comprises a soluble tungsten-containing catalyst that is, or is converted during the process to, a tungsten-containing anion that can be converted to tungstic acid at low pH, the pH of the aqueous medium is sufficiently reduced that tungstic acid is precipitated and then removed by a solids separation unit operation.

19. The process of claim 18 wherein the precipitated tungstic acid is reacted with base to form a soluble tungstate anion.

20. The process of claim 16 wherein a soluble tungsten-containing catalyst is used in the catalytic conversion, reduced tungsten-containing species form in the reaction zone, and the aqueous medium being recycled to the reaction zone is subjected to an oxidation unit operation to convert solid tungsten-containing species to soluble tungstate species.

21. The process of claim 20 wherein one or more of oxygen, ozone, peroxides, and soluble peracid and peroxyanion compounds are passed to the oxidation unit operation.

22. The process of claim 16 wherein organic acid is contained in the aqueous medium and at least a portion of the withdrawn aqueous medium is subjected to carboxylic acid hydrogenation conditions including the presence of a carboxylic acid hydrogenation catalyst and hydrogen at elevated temperature and pressure.

23. The process of claim 16 wherein at least one unit operation to enhance the catalytic process in the reaction zone comprises increasing the hydrogen partial pressure of the aqueous medium.

24. A catalytic process for producing a lower glycol of at least one of ethylene glycol and propylene glycol from a carbohydrate-containing feed comprising at least one of aldose- and ketose-yielding carbohydrate, said process comprising continuously or intermittently supplying the feed to a reaction zone containing an aqueous medium having therein catalyst for converting said carbohydrate to said glycol, said catalyst comprising heterogeneous hydrogenation or hydrogenolysis catalyst, and said aqueous medium being at catalytic conversion conditions including the presence of dissolved hydrogen, to produce a reaction product containing said glycol, wherein
(i) continuously or intermittently at least a portion of the aqueous medium is withdrawn from the reaction zone, said withdrawn aqueous medium containing heterogeneous catalyst, and
(ii) recycling at least a portion of the withdrawn aqueous medium from the reaction zone to the reaction zone, said recycled aqueous medium containing heterogeneous catalyst from the reaction zone, and prior to being introduced into the reaction zone, hydrogen is admixed with the recycling aqueous medium to provide a hydrogen-laden aqueous medium.

25. A catalytic process for producing a lower glycol of at least one of ethylene glycol and propylene glycol from a carbohydrate-containing feed comprising at least one of aldose- and ketose-yielding carbohydrate, said process comprising continuously or intermittently supplying the feed to a reaction zone containing an aqueous medium having therein a homogeneous, tungsten-containing retro-aldol catalyst and heterogeneous hydrogenation catalyst for converting said carbohydrate to said glycol, said aqueous medium being at catalytic conversion conditions including the presence of dissolved hydrogen, to produce a reaction product containing said lower glycol, wherein
(i) tungsten compound precipitates on the hydrogenation catalyst during the process;
(ii) continuously or intermittently at least a portion of the aqueous medium containing said hydrogenation catalyst is withdrawn from the reaction zone;
(iii) at least a portion of the withdrawn aqueous medium containing hydrogenation catalyst is subjected to at least one unit operation to remove a portion of the water and the lower glycol to a separated phase and provide a retained liquid phase containing at least about 10 mass percent of the lower glycol contained in the aqueous medium as withdrawn from the reaction zone and less than 10 volume percent water, and said hydrogenation catalyst wherein at least of the portion of the tungsten compound precipitated on the hydrogenation catalyst is solubilized;
(iv) maintaining the pH of the retained liquid phase from about 4 to 10 to maintain solubilized tungsten compound; and
(v) at least a portion of the retained liquid phase containing the solubilized tungsten compound is passed to the reaction zone.

26. The process of claim 25 wherein water is added to the retained liquid phase after step (a).

27. The process of claim 25 wherein the at least one unit operation comprises a vapor/liquid separation.
